(19) United States
(12) Patent Application Publication
(10) Pub. No.: US 2008/0220434 A1
(43) Pub. Date: Sep. 11, 2008

(54) Detection Of Molecule Proximity

(75) Inventor: James P. Thomas, New Albany, OH (US)

Correspondence Address: Casimir Jones, S.C., 440 Science Drive, Suite 203, Madison, WI, 53711, US

(73) Assignee: Perscitus Biosciences, LLC, Madison, WI (US)

(21) Appl. No.: 12/027,431

(22) Filed: Feb. 7, 2008

Related U.S. Application Data
(60) Provisional application No. 60/900,038, filed on Feb. 7, 2007.

(51) Int. Cl. C12Q 1/68

(52) U.S. Cl. 435/6

(57) ABSTRACT

The present invention provides methods, compositions and kits for identifying molecules such as proteins or nucleic acids that are found in proximity to each other in vitro or in vivo. For example, the present invention provides for the modification of one or more molecules that are complexed with, or in proximity to, a target biomolecule, wherein the modification of the one or more complexed or proximal molecules is detected.

The present application claims priority to U.S. Provisional Patent Application Ser. No. 60/900,038 filed Feb. 7, 2007, the entire disclosure of which is herein incorporated by reference in its entirety.

### FIELD OF THE INVENTION

The present invention provides methods, compositions and kits for identifying molecules such as proteins or nucleic acids that are found in proximity to each other in vitro or in vivo. For example, the present invention provides for the modification of one or more molecules that are complexed with, or in proximity to, a target biomolecule, wherein the modification of the one or more complexed or proximal molecules is detected.

### BACKGROUND OF THE INVENTION

The rapid determination of genomic sequences in species from man to fruit fly has promulgated one of the most daunting scientific challenges of the last century; the determination of the function of the myriad of proteins, nucleic acids, or other biomolecules encoded by these genetic sequences. Proteins are the active products of almost all genes, carrying out the primary functions of a cell in response to intracellular and extracellular signals. There are approximately 40,000 different proteins encoded by the human genome and many of these proteins exist in different potential forms as a result of post-translational modifications. The functions of the vast majority of these proteins are unknown. The identification and characterization of the interactions of a protein has emerged as one of the most studied research areas in the post-genomic era.

Although some proteins may perhaps perform their requisite activities in isolation, the overwhelming majority of proteins are expected to function in concert with other proteins in defined complexes and networks. Characterizing these protein-protein interactions represents a major challenge in bioscience research. Implications about a protein's function can be ascertained by the company it keeps. Protein-protein interactions can alter, for example, enzyme activity, allow for substrate channeling, create new allosteric sites for effector function, change substrate specificity, inactivate proteins, regulate transcription, or target a protein for degradation to name but a few of its potential myriad functions.

As protein-protein interactions are so important there are a multitude of methods to detect them. Each of the approaches has its own strengths and weaknesses, especially with regard to the sensitivity and specificity of the method. Co-immunoprecipitation is considered to be the gold standard assay for protein-protein interactions, especially when it is performed with endogenous (e.g., not overexpressed and not tagged) proteins. Typically, the protein of interest is isolated with a specific antibody, and western blotting subsequently identifies strong interacting partners to this protein. The yeast and/or mammalian two-hybrid systems investigate the interaction between artificial fusion proteins inside the nucleus of yeast or in the cytoplasm of a mammalian cell, respectively. This approach can identify binding partners of a protein in an unbiased manner. Tandem affinity purification (TAP) detects interactions within the correct cellular environment (e.g. in the cytosol of a mammalian cell) (Rigaut et al., 1999, Nat. Biotech. 17:1030-1032), however requires two successive steps of protein purification. Quantitative immunoprecipitation combined with knock-down (QUICK) relies on co-immunoprecipitation, quantitative mass spectrometry and RNA interference (RNAi). This method detects interactions among endogenous non-tagged proteins (Selbach and Mann, 2006, Nat. Methods. 3:981-983).

Protein interactions can also be detected using eTag™ Assays (Aclara Biosciences and Monogram Biosciences, U.S. Pat. Nos. 7,041,459, 7,037,654, 7,001,725, 6,955,874 and 6,949,347; incorporated herein by reference in their entireties). For example, the eTaq™ systems are used to show protein interactions by labeling proteins with an antibody conjugated to a fluorescent moiety. An additional antibody to the target protein is conjugated to a cleavage enzyme, which is also incorporated into the reaction. Once labeled, the reaction is exposed to light, followed by the photoactivated release of the cleavage enzyme (cleavase) that cleaves the fluorescent moiety away from the bound antibodies allowing for detection of the particular antibody bound protein by electrophoretic detection of the released fluorescent moiety. If proteins containing an antibody/fluorescent moiety bind to the target protein in the vicinity of the cleavage enzyme, the cleavage enzyme will release the fluorescent moiety and that protein will be indirectly detected due to the release of the fluorescent moiety.

However, fundamental flaws plague these techniques. For example, in the evaluation of two proteins it is unknown if further protein interactions are also present. It is not a sure thing that protein-protein interactions will survive purification. Purification and subsequent precipitation protocols are harsh and require a very stable protein-protein interaction to survive such isolation and purification conditions, conditions that do not exist in vivo. The two-hybrid systems are notorious for false positive results, which necessitate a second verification using, for example, co-immunoprecipitation. The eTaq™ system does not detect and identify unknown proteins, as the antibody/fluorescent moiety complexes need to be created so sequences of the proteins must be known. As well, the eTaq™ system requires that the proteins be in direct contact, or known binding partners, to each other. Also, proteins not in direct contact with the target protein are not detected, be they known or unknown. As such, current methodologies exclude identification of proteins that are not in physical contact with each other, and therefore do not identify proteins in a complex that may be associated with that complex, but not in physical contact with a target.

What are needed are compositions, systems and methods for studying complex biomolecular interactions and networks such that the potential for identifying all proximal biomolecules interacting in a complex or environs, regardless of degree of direct interaction with a target can be realized.

### SUMMARY OF THE INVENTION

The present invention provides methods, compositions and kits for identifying molecules such as proteins or nucleic acids that are found in proximity to each other in vitro or in vivo. For example, the present invention provides for the modification of one or more molecules that are complexed with, or in proximity to, a target biomolecule, wherein the modification of the one or more complexed or proximal molecules is detected.

In one embodiment, the present invention provides a binding partner (e.g., antibody, natural or synthetic ligand, an aptamer, small molecule, etc.) to a target biomolecule (e.g., protein, nucleic acid of interest, etc.). In some embodiments, the target is identified by using gene array technologies or similar technologies, wherein it is suggested that the target is an important component in a certain process. In some embodiments, the function of the target is unknown, whereas in other embodiments the function of the target is known and established. For example, a target could be a biomolecule associated with certain disease states and conditions such as cancer (e.g., breast, pancreatic, liver, lung, colon, skin, brain, etc.), neurodegenerative diseases (e.g., Alzheimer's, Parkinson's, sporadic amyotrophic lateral sclerosis, etc.), autoimmune diseases (e.g., AIDS, multiple sclerosis, Crohn's disease, systemic lupus erythematosus, etc.), aging, or inflammatory diseases (rheumatoid arthritis, osteoarthritis, arthritis, pulmonary diseases, asthma, etc.). For example, the processes associated with aging are starting to be elucidated. A current research focus is the identification of proteins and their interacting partners that are associated with this process. The proteins dihydropyrimidinase-like 2, alpha-enolase, dynamin-1, and lactate dehydrogenase have been identified as potentially important proteins (e.g., proteins of interest) associated with the aging process (Poon et al., 2006, Neurobiol. Aging 27:1010-1019; incorporated herein by reference in its entirety). Therefore, the use of one or more of these proteins as targets in the methods of the present invention allows a scientist to identify proteins that associate directly (e.g., complex with) and indirectly (e.g., in proximity to but not complexed with) with these targets (e.g., protein of interest) and helps in elucidating the processes associated with aging, as well as identifying therapeutic targets (e.g., for identifying molecules that enhance or disrupt associations between molecules). In some embodiments, targets are key proteins in a cellular metabolic pathway or a cascade of events that lead to and are involved in a particular cellular process or function.

In one embodiment, the present invention provides methods and kits for identifying molecules complexed with, or in proximity to, a target biomolecule wherein said complexed or proximal molecules are oxidized and modified (FIGS. 2A-D). In some embodiments, the oxidized, modified molecules are further complexed or modified with a compound capable of being directly or indirectly detected. For example, in some embodiments, the compound that derivatizes an oxidized molecule is dinitrophenylhydrazine (DNP) (FIG. 2E). In some embodiments, DNP is detected by binding to anti-DNP antibody followed by polyacrylamide gel electrophoresis and immunological analysis (e.g., ELISA, immunocytochemistry, immunohistochemistry, immunoblotting). In some embodiments, the detected molecules are further characterized by, for example, mass spectroscopy, nuclear magnetic resonance imaging (NMR), sequencing, or any other desired technique.

In one embodiment, the methods, compositions and kits of the present invention find utility in high throughput formats. For example, FIG. 3 shows an exemplary sample comprising a target biomolecule added to wells of a 96 well plate (e.g., further a 384 well, a 1536 well plate, etc.). In some embodiments, photosensitizer-conjugated antibodies specific to a target are added to their respective wells of the plate, and said antibodies are allowed to complex with their targets. The plate is then subjected to one or more pulses of visible light, at which point carbonyl reactive bonds, for example, are formed in the molecules complexed with, or proximal to, the target. In some embodiments, carbonyl groups thusly formed are derivatized with DNP, the samples are transferred to an anti-DNP coated 96 well plate, the plates are washed, and the bound molecules of interest are analyzed by Maldi-T of or LS-MS/MS.

In one embodiment, the methods and kits of the present invention find utility in detecting nucleic acid:protein interactions (FIG. 4). For example, a nucleic acid (e.g., oligonucleotide, DNA, RNA, etc.) is linked (e.g., via reactive amine groups) to a photosensitizer. The modified nucleic acid is incubated with a sample (e.g., nuclear extract, cytoplasmic extract, cell extract, cells) and subjected to visible light. Molecules complexed with, or in proximity to, the modified DNA are subsequently modified themselves to contain reactive groups by singlet oxygen, allowing for subsequent derivatization by, for example, the DNP hapten, followed by capture with anti-DNP antibodies and characterization of the molecule as previously described. The present invention also includes other embodiments described herein, or in view of knowledge in the art.

In one embodiment, the present invention provides a method for detecting molecules complexed with, or in proximity to, a target biomolecule comprising providing a sample with a target biomolecule, adding to said sample an activatable molecule for association with said biomolecule, applying an activator to said sample so as to activate said activatable molecule to provide modifications to molecules within proximity to said target biomolecule, and detecting said modifications to said molecules to identify molecules complexed with, or in proximity to, said target biomolecule. In some embodiments the sample is a cell lysate, cell extract, cell, tissue, environmental sample, or bodily fluid such as cerebrospinal fluid, urine, blood, plasma, serum, saliva, or bone marrow. In some embodiments, the target biomolecule is nuclear or cytoplasmic. In some embodiments, the target molecule is further from a mammal, a virus, or bacteria. In some embodiments, the target molecule from a mammal, a virus, or bacteria is a protein, a nucleic acid, a signal transduction component, a receptor, a transcription factor, a histone, an enzyme, a kinase, a phosphatase, a galactosidase, a nuclease, a protease, a polymerase, a transferase, a transcriptase, a ligase, a reporter enzyme, a protamine, a phosphoprotein, a mucoprotein, a chromoprotein, a lipoprotein, a nucleoprotein, a glycoprotein, a T-cell receptor, a proteoglycan, a cancer antigen, a tissue specific antigen, hormones, or a nutritional marker. In some embodiments, the target biomolecule from a mammal, a virus, or bacteria is DNA, cDNA, telomeric DNA, RNA, mRNA, hnRNA, miRNA, siRNA, dsRNA, or an oligonucleotide.

In one embodiment, the activatable molecule is a photosensitizer. In some embodiments, the activatable molecule is conjugated to a binding moiety wherein said binding moiety is in association with said target biomolecule. In some embodiments, the binding moiety is an antibody, a receptor, a ligand, or an aptamer. In some embodiments, the activator is activated by energy, light, or a chemical.

In one embodiment, modifications to molecules complexed with, or in proximity to, a target biomolecule comprise the creation of carbonyl groups, sulfur oxidation, tyrosine crosslinks, chlorination, nitrosation, hydroxylation, tryptophanyl modifications, hydroxyl derivatives of aliphatic amino acids, protein deamination, amino acid interconversions, amino acid oxidation adducts, glycoxidation adducts, cross-linking, aggregation, or peptide bond cleavage. In some embodiments, molecules in proximity to a target biomolecule are within at least 25 angstroms, at least 50 angstroms, at least 75 angstroms, at least 100 angstroms, at least 150 angstroms, at least 200 angstroms of the target biomolecule. In one embodiment, detecting modifications to molecules complexed with, or in proximity to, a target biomolecule comprise chemical detection, such as derivatization of a modification with dinitrophenylhydrazine, which is further captured by an antibody to dinitrophenylhydrazine and detected, for example, by an immunological assay (e.g., enzyme linked immunosorbent assay, immunohistochemistry, immunocytochemistry, immunoblotting). In some embodiments the modification molecules are detection by derivatization with a biotinylated compound, which is further captured with streptavidin and detected, for example, by colorimetry, fluorometry, or radiometry. In some embodiments, identifying the modified, captured molecules is performed by, for example, mass spectroscopy (e.g., Maldi-T of, LC-MS/MS), nuclear magnetic resonance imaging, or sequencing.

In one embodiment, detection of a modified molecule that is complexed with, or in close proximity to, a target biomolecule comprises reducing the modification with a reducing agent (e.g., DTT, BME), followed by biotinylation, capture with strepavidin, and chemical detection (e.g., colorimetry, spectrometry, radiometry) of the modified and reduced molecule.

In one embodiment, the present invention provides a kit comprising an activatable molecule, a compound reaction with carbonyl or sulfhydryl reactive groups, and a compound capable of capturing the reactive compound. In some embodiments, the kit further comprises a system for performing an enzyme linked immunosorbent assay.

### DESCRIPTION OF THE FIGURES

FIG. 1 shows an exemplary photosensitizer molecule conjugated to a monoclonal antibody (Mab).

FIG. 2 depicts an exemplary method for detecting molecules in proximity to a target molecule: A) the square is the target, and the cylinder and oval represent exemplary molecules in proximity to the target, B) a photosensitizer/monoclonal antibody conjugate binds to the molecule, C) upon application of light, the photosensitizer generates singlet oxygen (O₂), D) carbonyl bonds are created in the oxidized molecules, and E) carbonyl bonds react with DNP for detection of the molecules complexed with the target.

FIG. 3 shows an example of a high-throughput method for detection of biomolecules complexed with, or in proximity to, a target molecule. For example, a 96 well plate format is depicted which contains a complex biologic mixture to which is added a photosensitizer-conjugated antibody. After illumination of the reaction mixture, the biomolecules are oxidized, and the resultant carbonyl bonds are derivatized with DNP. The DNP labeled biomolecules are captured on a plate coated with an anti-DNP antibody. In this example, the captured biomolecules are characterized by Maldi-T of.

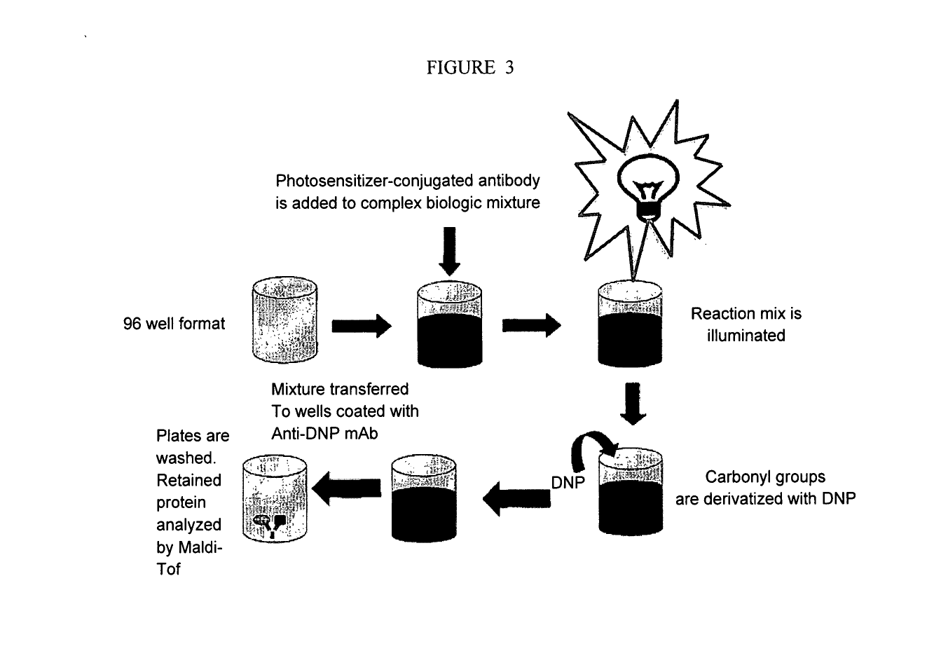

FIG. 4 is exemplary of using the compositions and methods for detecting molecules that complex with, or are in proximity to, a target nucleic acid molecule. A DNA molecule is conjugated with a photosensitizer molecule. Biomolecules are allowed to associate with the target DNA molecule and the sample is irradiated thereby causing oxidation of the complexed or proximal biomolecules. In this example, DNP is used to derivatize the carbonyl bonds of the complexed and proximal biomolecules, followed by capture of the labeled biomolecules on a surface coated with an antibody to DNP (anti-DNP).

FIG. 5 is exemplary of a biomolecule, in this case a protein that contains carbonyl bonds and is conjugated to biocytin hydrazide for capture with streptavidin.

FIG. 6 is exemplary of a target biomolecule that comprises a binding moiety Cys-X-X-Cys that is incorporated into the target protein for binding with an activatable molecule.

### DEFINITIONS

The term “epitope” as used herein refers to that portion of an antigen (e.g., protein or peptide) that makes contact with a particular antibody.

The terms “specific binding” or “specifically binding” refers to molecular interactions between one or more molecules, wherein one molecule recognizes and attaches to (e.g., binds) another molecule. For example, protein ligands recognize and bind to their receptors, enzymes recognize and bind to nucleic acid sequences, antibodies recognize peptide sequences and bind to those sequences. Therefore, in some embodiments molecules recognize biomolecular binding partners and bind to them thereby creating a biomolecular complex.

As used herein, the terms “non-specific binding” and “background binding” is the converse of “specific-binding”, and refers to molecular interactions that are not specific. Non-specific binding then refers to molecular interactions that are not dependent on the presence of a particular structure or sequence, and denotes the general binding and interaction of molecules.

As used herein, the term “oligonucleotide,” refers to a short length of single-stranded polynucleotide chain. Oligonucleotides are typically less than 200 residues long (e.g., between 15 and 100), however, as used herein, the term is also intended to encompass longer polynucleotide chains. Oligonucleotides are often referred to by their length. For example, a 24 residue oligonucleotide is referred to as a “24-mer”.

As used herein, the term “nucleic acid” refers to any nucleic acid containing molecule, including but not limited to, DNA (e.g., cDNA, genomic DNA, DNA fragments, etc.) or RNA (e.g., mRNA, hnRNA, miRNA, siRNA, dsRNA, etc.). The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl)uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyamino-methyl-2-thiouracil, beta-D-mannosylqueosine, 5′-methoxycarbonylmethyluracil, 5-methoxyuracil, 2-methylthio-N-6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

“Amino acid sequence” and terms such as “polypeptide” or “protein” are not meant to limit the amino acid sequence to the complete, native amino acid sequence associated with the recited protein molecule. Fragments thereof, be they functional or non-functional, are also encompassed by the aforementioned terms.

The term “native protein” is used herein to indicate that a protein does not contain amino acid residues encoded by vector sequences; that is, the native protein contains only those amino acids found in the protein as it occurs in nature. A native protein may be produced by recombinant means or may be isolated from a naturally occurring source, or may be found in a biological environment either in vitro or in vivo.

As used herein the term “portion” when in reference to a protein (as in “a portion of a given protein”) refers to fragments of that protein. The fragments may range in size from four amino acid residues to the entire amino acid sequence minus one amino acid. Portions of a protein may be functional or non-functional.

As used herein, the term “in vitro” refers to an artificial environment and to processes or reactions that occur within an artificial environment. In vitro environments can consist of, but are not limited to, test tubes (e.g., cell lysates and extracts) and cell culture (e.g., in a culture dish or tissue explants or samples). The term “in vivo” refers to the natural environment (e.g., an animal or a cell) and to processes or reactions that occur within a natural environment.

As used herein, the term “sample” is used in its broadest sense. In one sense, it is meant to include a specimen or culture obtained from any source, as well as biological and environmental samples. Biological samples may be obtained from animals (including humans) and encompass fluids (e.g., saliva, urine, cerebrospinal fluid, blood, plasma, serum, etc.), solids, tissues. Biological samples include cells, cellular lysates, extracts and the like. Environmental samples include environmental material such as surface matter, soil, water, and industrial samples. Such examples are not however to be construed as limiting the sample types applicable to the present invention.

As used herein, the term “photosensitizer” is used to define a molecule that absorbs radiation of one or more defined wavelengths and subsequently utilizes the absorbed energy to carry out a chemical process. In some embodiments, a photosensitizer is a molecule that, upon administration of visible light (e.g., around 400 nm to around 700 nm), oxidizes organic compounds, for example proteins, with participation of singlet oxygen. However, a skilled artisan will recognize that any wavelength of light can activate a photosensitizer, and the light wavelength necessary to activate a photosensitizer is specific to the structure of the photosensitizer. The present invention in not limited by the photosensitizer, nor the wavelength for its activation.

As used herein, the term “activatable molecule” refers to a molecule that, upon application of an activator, is activated to perform a certain function. For example, an activatable molecule can be a photosensitizer, such that application of light activates (e.g., energizes) the photosensitizer, in the present application producing singlet oxygen. Iron is an additional example of an activatable molecule.

### DETAILED DESCRIPTION OF THE INVENTION

Most biomolecular complexes exist as multiple molecules that are either directly (e.g., complexed with) or indirectly (e.g., in proximity to) associated with a target biomolecule. The vast majority of associated molecules in a biomolecular complex have not been identified, or are not readily identifiable using methods and systems currently available. Available methods and systems are limiting and are not amenable to identifying molecules in a complex that do not directly bind to a target of interest, and therefore many molecules that interact for performing a particular process in a cell are missed and never identified as important components of a cellular process. The compositions and methods of the present invention recognize molecular interactions that exist in biomolecular complexes, that have to date been missed by current methodologies. The compositions and methods of the present invention are described in exemplary embodiments provided below. However, the present invention is not limited to these embodiments, and a skilled artisan will recognize additional embodiments applicable to the present invention.

The present invention provides compositions, methods and kits for identifying molecules (e.g., proteins, nucleic acids, small molecules, etc.) that are found in proximity to each other in vitro or in vivo. In one embodiment, the present invention provides a target biomolecule that is in association with an activatable molecule (e.g., photosensitizer molecule, iron chelator molecule). In some embodiments, the activatable molecule is conjugated directly to the target biomolecule, or indirectly to the target biomolecule. In some embodiments, the indirect attachment of the activatable molecule to the target biomolecule is such that the activatable molecule is first conjugated to a second molecule (e.g., antibody, peptide, nucleic acid, small molecule, etc.), and that second molecule (e.g., antibody, peptide, nucleic acid, small molecule, etc.) attaches to the target biomolecule. In some embodiments, the activatable molecule is activated by exposure to light. In some embodiments, the light used to activate the activatable molecule is visible light (e.g., wavelengths between around 400-700 nm). If the activatable molecule is a light activated molecule like a photosensitizer, it is further not limited to its wavelength of activation, indeed photosensitizers that are activated by ultraviolet (e.g., wavelengths between 300-400 nm) and infrared (e.g., wavelengths between 700-800 nm) light are also useful in the present invention. In some embodiments, the sphere of reactivity of a photosensitizer activatable molecule is increased or decreased by augmenting the time of irradiation, by increasing the number of photosensitizers linked to an antibody, by including a singlet oxygen quencher or scavenger (e.g., azide, polyenes, carotenoids, vitamin E, vitamin C, amino acid-pyrrole N-conjugates of tyrosine, histidine, and glutathione, and the like) in a reaction, or other like approaches. In some embodiments, the activatable molecule is chemically or electrically activated.

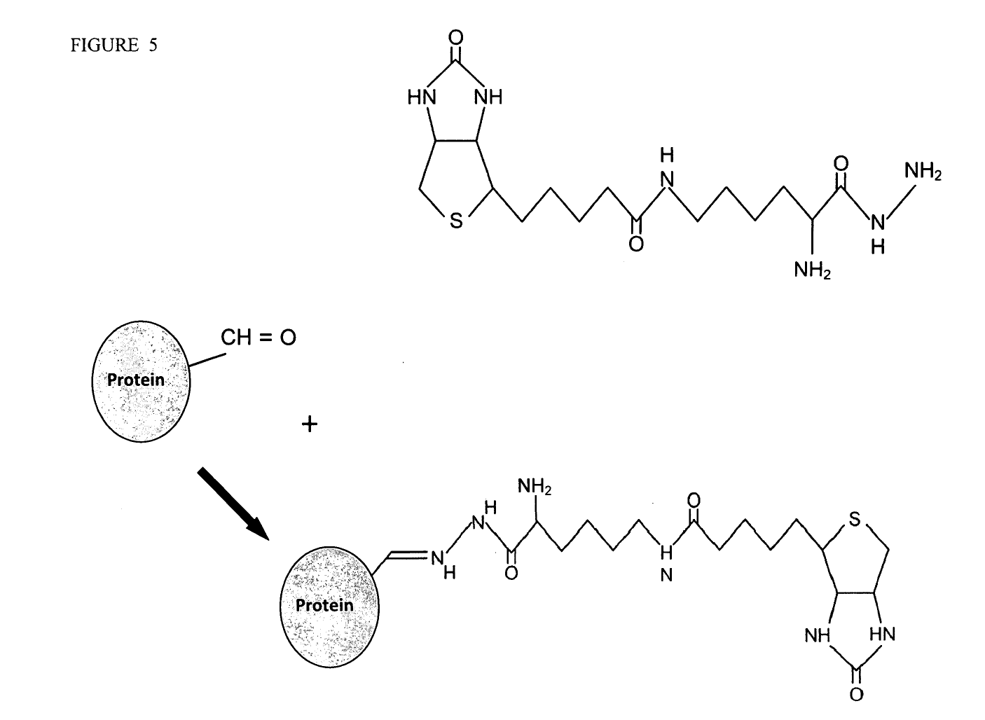

In some embodiments, activation of the activatable molecule allows for modification of molecules that are complexed with, or in proximity to, the target biomolecule. In some embodiments, the activated molecule is capable of producing singlet oxygen that modifies that target biomolecule and molecules complexed with, or in proximity to, the target biomolecule. In some embodiments, modification of the molecules includes, for example, the formation of reactive carbonyl groups. In some embodiments, the carbonyl groups are derivatized with DNP. In some embodiments, the DNP labeled molecules are captured and purified away from reaction components by, for example, anti-DNP antibodies coated on a substrate (e.g., slide, plate, beads, membrane, etc.), followed by washing of the substrate to remove the reaction components and non-bound species. In some embodiments, the labeled molecules are separated by electrophoresis. In some embodiments, the carbonyl groups are derivatized with a biotinylating compound, such as biocytin hydrazide or other biotin derivative capable of binding reactive carbonyl or sulfhydryl groups (FIG. 5). In some embodiments, the biotinylated biomolecules are captured and purified away from reaction components by, for example, a streptavidin coated substrate (e.g., slide, plate, beads, membrane, etc.), followed by washing of the substrate to remove the reaction components and non-bound species. In some embodiments, the captured biotinylated molecules are detected by colorimetric, fluorimetric, or radiometric detection methods.

In some embodiments, the modified molecules contain disulfide bonds upon exposure to an activated molecule (e.g., activated photosensitizer). In some embodiments, the disulfide bonds are further reduced by a reducing agent such as, for example, DTT or βME, thereby creating reactive sulfhydryl groups in the molecules. In some embodiments, the sulfhydryl groups are derivatized with, for example, a biotinylating compound, and captured and characterized as previously described.

In some embodiments, the captured and purified molecules are characterized by, for example, mass spectroscopy, sequencing, NMR, or other methods known to a skilled artisan. As such, the compositions and methods of the present invention allow for the identification of molecules that are complexed with, or in proximity to, a target biomolecule.

In one embodiment, the present invention provides for the detection and identification of molecules that complex with, or are in proximity to, a target biomolecule. In some embodiments, the target biomolecule is, for example, a protein, a nucleic acid, a signal transduction component, a receptor, a transcription factor, a histone, an enzyme, a kinase, a phosphatase, a galactosidase, a nuclease, a protease, a polymerase, a transferase, a transcriptase, a ligase, a reporter enzyme, a protamine, a phosphoprotein, a mucoprotein, a chromoprotein, a lipoprotein, a nucleoprotein, a glycoprotein, a T-cell receptor, a proteoglycan, a cancer antigen, a tissue specific antigen, hormones, a nutritional marker, DNA, cDNA, telomeric DNA, RNA, mRNA, hnRNA, miRNA, siRNA, dsRNA, or an oligonucleotide.

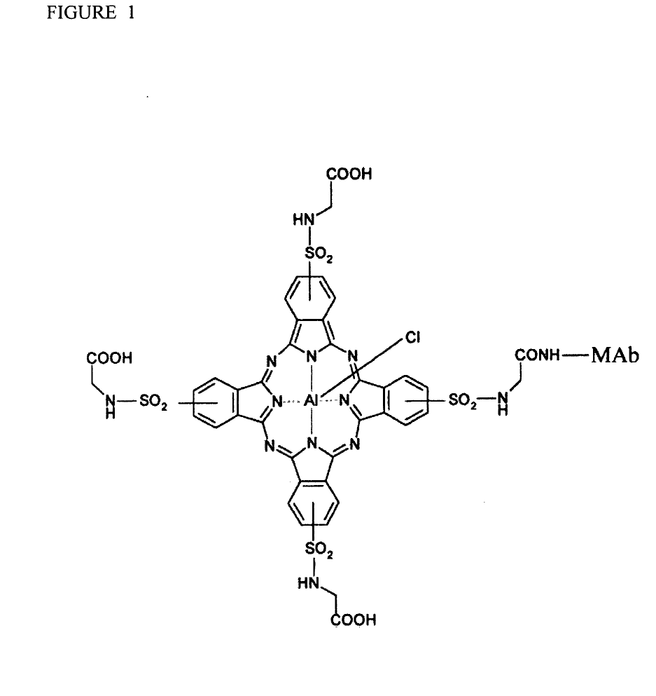

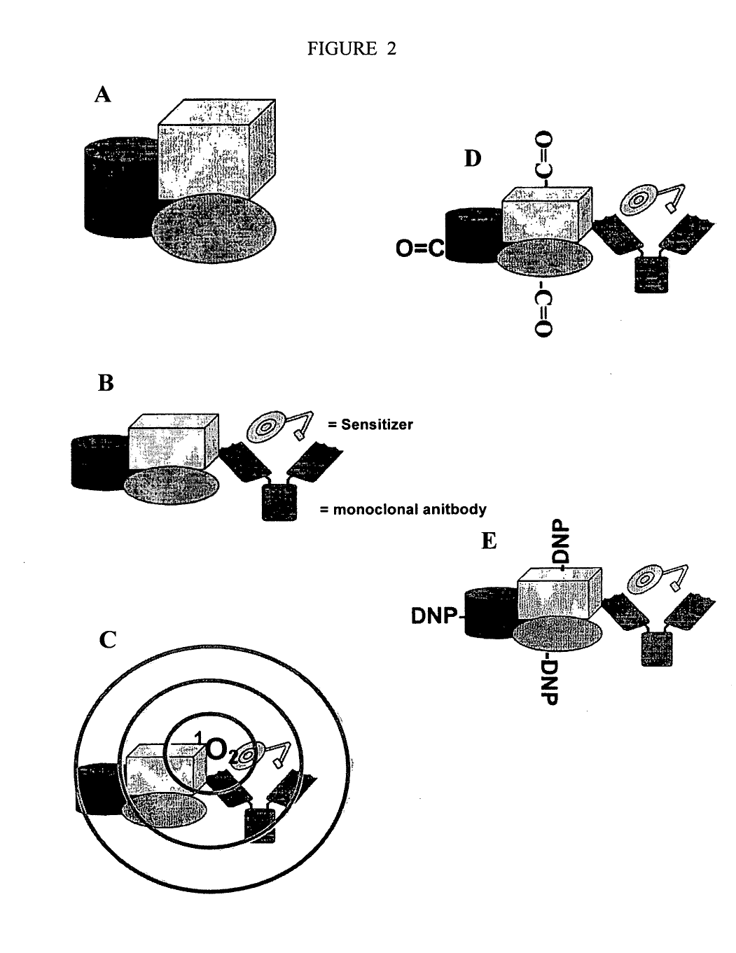

In one embodiment, the target biomolecule is conjugated to an activatable molecule either directly or indirectly. In some embodiments, the activatable molecule is complexed directly to the target biomolecule. In some embodiments, the activatable molecule is first conjugated to a binding moiety, such that the binding moiety is directly bound to the target biomolecule. Examples of binding moieties include, but are not limited to, antibodies (e.g. monoclonal or polyclonal), receptors, ligands, and aptamers. For example, FIG. 2 is exemplary of a method of the present invention wherein a photosensitizer (e.g., activatable molecule), such as found in FIG. 1, is conjugated with an antibody (e.g., binding moiety), and the antibody binds to the protein of interest (e.g., target biomolecule). As such, in some embodiments, the present invention provides antibodies that target biomolecules, wherein said antibodies are conjugated to an activatable molecule, such as a photosensitizer molecule. In some embodiments, the present invention provides photosensitizer conjugated monoclonal antibodies that specifically bind to a target biomolecule. It is contemplated that an antibody against a target may be a monoclonal or polyclonal antibody as long as it can recognize the target biomolecule. However, monoclonal antibodies are preferred. Antibodies are produced, for example, by using a target, or fragment thereof, as the antigen according to conventional antibody or antiserum preparation processes as described in Harlow & Lane, 1988, Antibodies: A Laboratory Manual, Cold Spring Harbor Laboratory Press, pp. 726 (incorporated herein by reference in its entirety). In some embodiments, the antibody conjugated to an activatable molecule such as a photosensitizer molecule is a secondary antibody (e.g., goat anti-mouse, goat anti-rabbit, horse anti-mouse, etc.).

In one embodiment, an antibody is conjugated to a photosensitizer molecule capable of oxidizing organic molecules by producing singlet oxygen (Vrouenraets et al., 1999, Cancer Res. 59:1505-1513; Vrouenraets et al., 2002, Int. J. Cancer 98:793-798; incorporated herein by reference in their entireties). When a photosensitizer molecule is irradiated with light of a particular wavelength, the photosensitizer is converted to an energized form that reacts with oxygen such that, upon decay of the photosensitizer to the non-energized state, singlet oxygen is produced. An example of a photosensitizer molecule useful in the present invention can be found in FIG. 1. Properties and selection of photosensitizers can be found in, for example, Turro, 1991, Molecular Photochemistry, University Science Books, Baumstark, 1983, Singlet Oxygen Vol. II, CRC Press, Inc., Boca Raton Fla., and Wasserman and Murray, 1979, Singlet Oxygen, Academic Press; incorporated herein by reference in their entireties. However, the present invention is not limited to any particular photosensitizer molecule. In some embodiments, the photosensitizer molecule is conjugated to compositions such as an antibody, peptide, nucleic acid, small molecule, or functional equivalents thereof that are capable of recognizing and binding to a target biomolecule. In some embodiments, the photosensitizer molecule is energized by light to produce singlet oxygen. Other examples of photosensitizer molecules include, but are not limited to, rose bengal (Nowakowska et al., 2001, Pure Appl. Chem. 73:491-495), hypocrellin A, hypocrellin B, hyperacin, halogenated derivatives of fluorescein dyes, merocyanine 540, methylene blue, 9-thioxanthone, chlorophylls, phenalene, protoporphyrin, benzyporphryin A monacid, tetraphenylporphyrin, halo genated derivatives of rho damine dyes, metallo-porphyrins, phthalocyanines, naphthalocyanines, texaphryin-type macrocycles, hematoporphyrin, 9,10-dibromoanthracene, benzophenone, chlorine e6, perylene, and benzoporphyrin B monacid (Turro, N.J., 1991, Molecular Photochemistry, University Science Books, Ullmann et al., 1994, Proc. Natl. Acad. Sci. 91:5426, Strong et al., 1994, Ann. New York Acad. Sci. 745:297, Martin et al., 1990, Meth. Enz. 186:635, Yarmush et al., 1993, Crit. Rev. Therap. Drug Carrier Syst. 10:197, Wohrle, 1991, Chimia 45:307, and U.S. Pat. Nos. 7,049,110, 6,887,862, 6,610,298, 6,251,581, 5,763,602, 5,709,994, 5,536,834, 5,516,636, 5,340,716; all references and patents are incorporated herein by reference in their entireties). Photosensitizers useful in the present invention are preferentially energized upon irradiation with visible light (wavelengths around 400 nm to around 700 nm). However, the present invention is not limited to the wavelengths used, and photosensitizers with optimal wavelength excitation in the ultraviolet (around 300 to 400 nm) and infra-red (around 700 to 800 nM) ranges also find utility as photosensitizers in the methods and kits of the present invention.

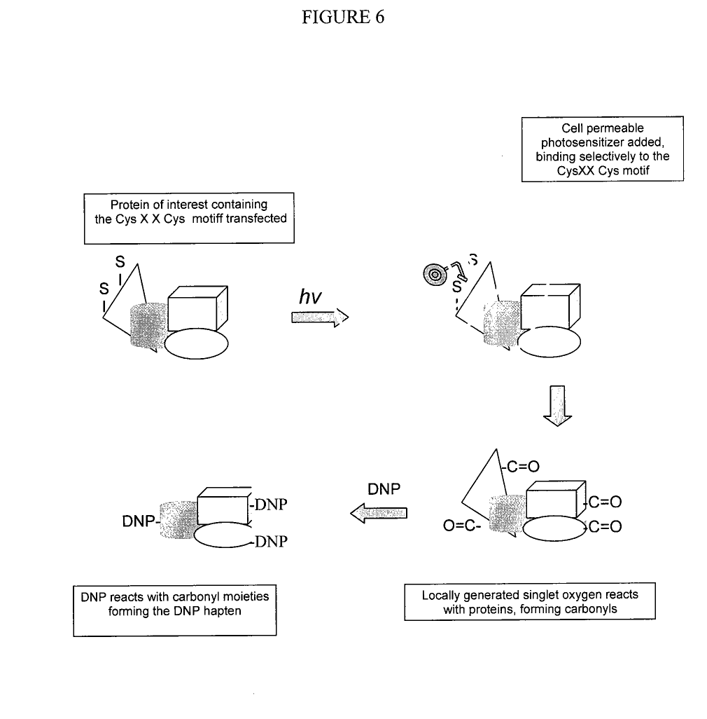

In one embodiment, the activatable molecule is a biarsenical membrane permeant photosensitizer or analogs thereof. For example, the compound ReAsH (Resorufin Arsenical Hairpin) is a resorufin derivatized photosensitizer molecule containing two arsenic substitutents that produces singlet oxygen. The compound FlAsH (Fluorescein Arsenical Hairpin) is a fluorescein derivatized photosensitizer molecule containing two arsenic substitutents that produces singlet oxygen. The FlAsH and ReAsH arsenic moieties bind to a tetracysteine motif, Cys-Cys-X-X-Cys-Cys wherein X is any noncysteine amino acid (Bulina et al., 2006, Nat. Biotech. 24:95-99; Adams et al., 2002, J. Am. Chem. Soc. 124:6063-6076, incorporated herein by reference in their entireties). As such, in some embodiments, the present invention provides for a target biomolecule comprising the motif Cys-Cys-X-X-Cys-Cys wherein X is any noncysteine amino acid. In some embodiments, the tetracysteine motif is cloned into a protein of interest (e.g. target biomolecule) such that normal protein function is maintained. For example, the tetracysteine motif is incorporated into the target biomolecule at the N or C terminus using methods known to those skilled in the art (DNA cloning as described, for example, in Ausubel et al., Current Protocols in Molecular Biology). The cloned DNA comprising the target protein with the tetracysteine motif is expressed in a cell, for example, in vivo, ex vivo, or in vitro using known methodologies (e.g., transfection using calcium phosphate precipitation, lipids, electroporation, etc.). As the biarsenical compounds are cell permeant, ReAsH, FlAsH, or analogs thereof are added to the experiment and complexation with the tetracysteine motif occurs. Light is applied to the reaction thereby activating ReAsH, FlAsH, or analogs thereof, followed by production of singlet oxygen that modifies the molecules complexed with, or in proximity to, the target biomolecule, which is then derivatized with, for example, DNP or a biotinylated compound and captured, detected and characterized as described herein (FIG. 6).

In one embodiment, the activatable molecule is iron (e.g., iron salt, iron oxide, iron chelates, etc.). An iron molecule, in the presence of a reducing superoxide radical O₂— and hydrogen peroxide, produces free hydroxyl reactive groups (OH— groups) (Halliwell, 1982, Biochem. J. Lett. 205:461; incorporated by reference herein in its entirety) thereby oxidizing proteins. In some embodiments, binding moieties to target biomolecules are conjugated to iron containing molecules (e.g., iron salt, iron chelator, iron oxide, etc.). Upon binding of the binding moiety to the target biomolecule, hydrogen peroxide and/or a reducing agent capable of generating O₂— is added to the biological environment, thereby allowing for the generation of free hydroxyl reactive groups on the molecules complexed with, or in proximity to, the target biomolecule. In some embodiments, the molecules containing free hydroxyl reactive groups are detected by, for example, HPLC using an aromatic hydroxylation assay (Kaur and Halliwell, 1994, Anal. Biochem. 220:11-15, incorporated herein by reference in its entirety), a deoxyribose assay (Gutteridge and Halliwell, 1988, Biochem. J. Lett. 253:932-33, incorporated herein by reference in its entirety), or other assay for detecting free hydroxyl radicals.

In one embodiment, the photosensitizer-conjugated antibody is added to a biological environment, either in vivo or in vitro, comprising the target. In some embodiments, the biological environment (e.g. cell lysates or extracts, cells, tissues, whole animal systems, etc.) is further exposed to one or more pulses of light. The present invention is not limited to a particular mechanism. Indeed, an understanding of the mechanism is not necessary to practice the present invention. Nonetheless, it is contemplated that one or more pulses of light activates the photosensitizer thereby producing singlet oxygen which diffuses a limited distance from its origin in the biomolecular complex (Krasnovsky, 1998, Membr. & Cell Biol. 12:665-690; Deadwyler et al., 1997, Photochem. & Photobiol. 65:884-894; incorporated herein by reference in their entireties). It is contemplated that the singlet oxygen diffuses at least 25 nm, at least 50 nm, at least 75 nm, at least 100 nm, at least 150 nm, at least 200 nm from the site of production. Diffusion distance is limited by, for example, the decay of the singlet oxygen and reaction with the biomolecules. However, diffusion distance can be controlled by, for example, the inclusion of a singlet oxygen scavenger (e.g., azide) in the biological environment.

The singlet oxygen, upon diffusion, oxidizes the molecules complexed with, or in close proximity to, the target, wherein molecules not in the vicinity of the target are not oxidized. Oxidation of molecules leads to different modifications. For example, modifications to molecules undergoing oxidation can result in sulfur oxidation (e.g. cysteine disulfides, mixed disulfides (e.g., glutathiolation, methionine sulfoxide), creation of protein carbonyls (e.g. at protein side chain aldehydes and ketones), tyrosine crosslinks, chlorination, nitrosation and hydroxylation, tryptophanyl modifications, hydroxyl derivatives of aliphatic amino acids, protein deamination, amino acid interconversions (e.g., H is to Asn), amino acid oxidation adducts (e.g., p-hydroxyphenylacetaldehyde), glycoxidation adducts (e.g., carboxymethyllysine) and general cross-linking, aggregation, and peptide bond cleavage. Directly or indirectly detectable molecule modifications find utility in the present invention.

For example, carbonyl (e.g., C═O) bonds created in oxidized molecules are susceptible to derivitization by additional compounds, such as dinitrophenylhydrazine (DNP), biocytin hydrazide (e.g., EZ-LINK biocytin hydrazide, Pierce) and tritiated sodium borohydride (NaB³H₃), thereby rendering the oxidized molecule directly or indirectly detectable (e.g., fluorescence, luminescence, calorimetric, radiometric, spectroscopy). Dinitrophenylhydrazine is a well-characterized hapten detectable using commercially available antibodies raised to DNP (Upstate Cell Signaling Solutions, Inc., OXYBLOT Protein Oxidation Detection Kit; Casinu et al., 2002, J. Clin. Once. 20:3478-3483; Tezel et al., 2005, Inv. Opthal. & Vis. Sci. 46:3177-3187; incorporated herein by reference in their entireties). Molecules complexed with, or in proximity to, the target can be identified by adding, for example, DNP to the photooxidized sample with subsequent detection using anti-DNP antibodies. In some embodiments, molecules are separated using one or two dimensional polyacrylamide gel electrophoresis (1D or 2D PAGE), and visualized (Yan et al., 1998, Anal. Biochem. 263:67-71, incorporated herein by reference in its entirety). In some embodiments, immunological assay methodologies (e.g., enzyme linked immunosorbent assays (ELISA), immunohistochemistry, immunocytochemistry, immunoblotting) (Shacter et al., 1994, Free Radic. Biol. Med. 17:429-437; Buss et al., 1997, Free Radic. Biol. Chem. 23:361-366; Smith et al., 1998, J. Histochem. Cytochem. 46:731-735; Shacter, 2000, Meth. Enzymol. 319:428-436; Tezel et al., 2005; incorporated herein by reference in their entireties) using anti-DNP or another detection antibody find utility in methods and kits of the present invention by identifying DNP modified molecules, or other modified molecules. Methods for separating molecules of interest can also include purification columns. In some embodiments, molecules of interest are characterized by, for example, mass spectrometry (e.g., matrix assisted laser desorption ionization time-of-flight mass spectrometry (MALDI-T of) or liquid chromatography tandem mass spectrometry (LC-MS/MS)) (Tezel et al., 2005; Lennon, 1997, Matrix Assisted Laser Desorption Ionization Time-of-flight Mass Spectrometry at www.abrf.org/ABRFNews/1997/June1997/jun97lennon.html; incorporated herein by reference in their entireties), nuclear magnetic resonance imaging, or sequencing.

Oxidized molecules created by practicing the methods of the present invention can also be, for example, biotinylated by reacting the carbonyl groups with biocytin hydrazide and capturing with streptavidin on a streptavidin-coated plate, membrane or coated beads. Biotinylated proteins are characterized, for example, as previously described by using, for example, LC-MS/MS techniques (Soregahan et al., 2003, Pharm. Res. 20:1713-1720, incorporated herein in its entirety).

In one embodiment, oxidized molecules contain disulfide bonds at cysteine residues in an amino acid due to oxidation by the photosensitizer and can be detected, isolated, and characterized. For example, the disulfides are reduced to reactive sulfhydryl groups by addition of a reducing agent (e.g., β-mercaptoethanol (βME), dithiothreitol (DTT), etc.) to the sample. Once reduced, the reactive sulfhydryl groups are free to react with biotin molecules, and the molecules are captured and characterized (Shacter, 2000, Drug Metab. Rev. 32:307-326; Makmura et al, 2001, Antiox. & Redox. Sign. 3:1105-1118; incorporated herein by reference in their entireties). However, the present invention is not limited by the methods used for detection, isolation, and characterization of oxidized molecules and those skilled in the art will recognize additional processes and protocols for detecting, isolating and characterizing oxidized molecules.

In one embodiment, the present invention provides a binding moiety that is a first antibody (e.g., primary antibody), complexed to the target in a sample. The primary antibody can be either monoclonal or polyclonal. In some embodiments, a second binding moiety, such as a second antibody (e.g., secondary antibody) conjugated to an activatable molecule, such as a photosensitizer, is added to the sample, such that the secondary antibody recognizes and binds to the primary antibody. In some embodiments, the secondary antibody is raised to recognize monoclonal antibodies, for example goat anti-mouse, or horse anti-mouse. In some embodiments, the secondary antibody is raised to recognize polyclonal antibodies, for example goat anti-rabbit or horse anti-rabbit. However, the present invention is not limited to the animal used to create the polyclonal antibody, nor is it limited in the animal used to raise the secondary antibody. A skilled artisan would understand that all that is required to practice the methods of the present invention are that the secondary antibody recognize and bind the primary antibody.

In one embodiment, the activatable molecule/target complex is added to cells in vivo. In some embodiments, the complex comprises an antibody that binds to a receptor on the cell surface that allows internalization of the complex into a cell. In some embodiments, the complex comprises a peptide or protein that is recognized by a receptor or other signal structure on the cell surface that allows internalization. For example, a target molecule can be conjugated with, or engineered to express (e.g., fusion protein), a peptide sequence that serves as a ligand to a cell surface receptor. For example, an RGD peptide that is recognized by integrins on the cell surface can be engineered into a molecule, or complexed with a molecule, for cell internalization (Ruoslahti, 1996, Annu. Rev. Cell. Biol. 12:697; incorporated herein by reference in its entirety). A ligand that recognizes a cell surface receptor is conjugated to the target biomolecule complex, thereby allowing for internalization into a cell. For example, concanavalin A, transferrin, and numerous hormones and growth factors (e.g., insulin, epidermal growth factor, calcitonin, prolactin, etc.) are recognized by cell surface receptors and internalized into a cell (Alberts et al, Molecular Biology of the Cell, Garland publishing, N.Y., Third Edition, 1994, incorporated herein by reference in its entirety). Viral fragments (e.g., adenovirus, lentivirus, rhinovirus, rous sarcoma virus, Semliki Forest virus, Herpes virus, etc.) that bind to cell surfaces and are internalized are complexed with the activatable molecule/target molecule complex for cell internalization (Rossman, 1994, Pro. Sci. 10:1712; Huang et al., 1996, J. Virol. 70:4502; incorporated herein by reference in their entireties). Such incorporation of internalization molecules into a complex targets specific cell types (e.g., target cancer cells, endothelial cells, pancreatic cells, airway epithelial cells, white blood cells, etc.) or generally targets cells such that the complexes are internalized into a wide range of cell types. The present invention further provides for target nucleic acids internalized by cells. For example, nucleic acids comprising active groups for complexing with an activatable molecule are internalized into cells as, for example, naked nucleic acids (e.g., DNA, RNA, oligonucleotides, etc.), or by using a variety of transfection means such as cationic lipids, DEAE-Dextran, calcium phosphate precipitation, electroporation and the like as found in Ausubel et al, Current Protocols in Molecular Biology (incorporated herein by reference in its entirety). The present invention is not limited by the method used for internalization of the activatable molecule/target complex into a cell, and a skilled artisan will recognize other methods and compositions that are applicable for internalization of molecules (e.g., small molecules, proteins, nucleic acids, etc.) into a cell.

In some embodiments, the target molecule/activatable molecule complex (e.g., protein, nucleic acid) is added to cells ex vivo. For example, cells or tissues are removed from a subject and explanted to an environment (e.g., tissue culture dish or other sterile substrate) that allows for continued growth and experimentation (e.g., the explanted material is bathed in culture media with requisite factors and compositions optimal for tissue growth). Explanted cells or tissues are exposed to activatable molecule/target protein or nucleic acid complexes for internalization of the complexes as previously described, for example. Alternatively, ex vivo treated cells and tissues can be transplanted into the same, or different subject (e.g., human explanted cells or tissues transplanted into mice or rats) allowing for ex vivo internalization of complexes followed by in vivo environmental conditions.

In one embodiment, the present invention provides for methods and kits for detecting and determining molecules complexed with, or in proximity to, a target in a sample. In some embodiments, said target is conjugated with a target specific antibody that is further complexed with a photosensitizer molecule. In some embodiments, the antibody conjugated to the target is a primary antibody, and a secondary antibody complexed to a photosensitizer molecule is added to the sample such that the secondary antibody recognizes and binds said primary antibody conjugated to the target. In some embodiments, said target and photosensitizer complexed antibody are both present in a sample. In some embodiments, said sample containing said target and said photosensitizer complexed antibody are exposed to one or more bursts of light. In some embodiments, said bursts of light activate said photosensitizer molecule with a resultant release of singlet oxygen. In some embodiments, the release of singlet oxygen oxidizes molecules complexed with, or in proximity to, said target (e.g., in the sphere of reactivity) in addition to said target. In some embodiments, said oxidized molecules are labeled, isolated, and further characterized.

In one embodiment, the present invention provides kits for performing the methods as described herein. In some embodiments, kits provide an activatable molecule that will oxidize molecules (e.g., photosensitizer molecule, etc.) In some embodiments, kits provide an activatable molecule that is conjugated to a binding moiety (e.g., antibodies (monoclonal or polyclonal), receptors, ligands, aptamers, etc.) that recognizes a target biomolecule (e.g., a protein, a nucleic acid, a signal transduction component, a receptor, a transcription factor, a histone, an enzyme, a kinase, a phosphatase, a galactosidase, a nuclease, a protease, a polymerase, a transferase, a transcriptase, a ligase, a reporter enzyme, a protamine, a phosphoprotein, a mucoprotein, a chromoprotein, a lipoprotein, a nucleoprotein, a glycoprotein, a T-cell receptor, a proteoglycan, a cancer antigen, a tissue specific antigen, hormones, a nutritional marker, DNA, cDNA, telomeric DNA, RNA, mRNA, hnRNA, miRNA, siRNA, dsRNA, oligonucleotide etc.). For example, kits comprise a photosensitizer labeled antibody (e.g., primary or secondary) that binds to a particular target biomolecule of interest, or a primary antibody bound to a target biomolecule of interest. In some embodiments, kits provide a compound (e.g., DNP, biotinylating compound, tritiated reagents, etc.) that will react with reactive groups (e.g., carbonyl groups, sulfhydryl groups, etc.). For example, kits comprise compounds such as DNP or a biotinylating compound that binds reactive groups in molecules that complex with, or are in proximity to, a target biomolecule that have been modified by an activatable molecule.

In some embodiments, kits comprise compounds that capture or immobilize compounds that bind to reactive groups. Antibodies raised to a reactive group binding compound and streptavidin are exemplary of capture or immobilization compounds that are themselves immobilized (e.g., on slides, plates, beads, membranes, etc.). In some embodiments, kits also contain detection systems for detecting the immobilized molecules that are complexed with, or in proximity to, a target biomolecule. Exemplary systems for detection include, but are not limited to, enzyme linked immunosorbent assays, immunohistochemistry, immunocytochemistry, immunoblotting, binding assays, and other assays for detection using colorimetry, fluorimetry, or radiometry. In some embodiments, kits of the present invention contain buffers, reagents, solutions, control reactions, and the like deemed important or necessary for performing the methods as described herein. In some embodiments, kits contain instructions for users which include, but are not limited to, methods for performing the present invention as described herein as well as adaptations of optimization of the methods. In some embodiments, kits of the present invention are adaptable by the user. For example, a user can increase or decrease the sphere of reactivity (e.g., oxidation by photosensitizer) by augmenting the time of irradiation, by increasing the number of photosensitizers linked to an antibody, or by including a singlet oxygen quencher (e.g., azide, polyenes, carotenoids, vitamin E, vitamin C, amino acid-pyrrole N-conjugates of tyrosine, histidine, and glutathione, and the like, (Beutner et al., 2000, Meth. Enzymol. 319: 226; incorporated herein by reference in its entirety)) in a reaction. As such, instructions included in the kit, in some embodiments, guides the user in optimization and adaptation of the kit components for user defined purposes.

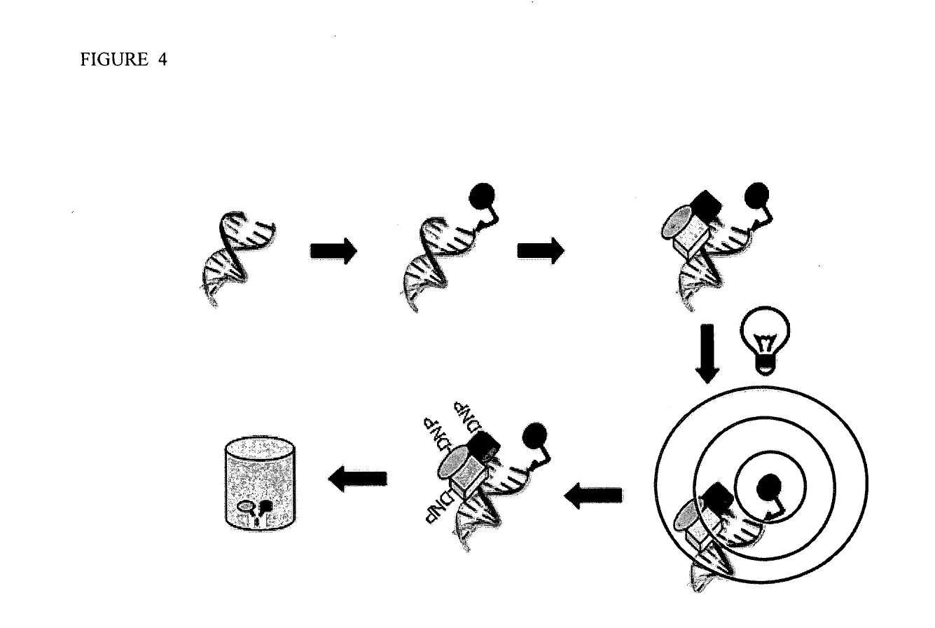

In one embodiment, the present invention provides methods and kits useful in identifying and characterizing molecules that complex with, or are in proximity to, a target biomolecule that is a nucleic acid. In one embodiment, the methods and kits detect nucleic acid:protein interactions (FIG. 4). For example, a nucleic acid (e.g., oligonucleotide, DNA, RNA, etc.) is conjugated to a photosensitizer molecule by linkage with reactive amine or carboxyl groups. The photosensitizer/nucleic acid conjugate is added to and incubated with a sample under conditions such that molecules that would normally associate with said nucleic acid are allowed to do so. The reaction mixture is subsequently subjected to visible light wherein the photosensitizer produces singlet oxygen. Molecules complexed with, or in proximity to, the target nucleic acid are modified themselves to contain reactive groups (e.g., carbonyl groups, sulfhydryl groups, etc.) by the singlet oxygen (or subsequent reduction of disulfide bonds into sulfhydryl reactive groups by reducing agents, etc.), allowing for subsequent derivatization by, for example, DNP hapten or biotinylating compounds, followed by capture with anti-DNP antibodies or streptavidin and characterization of the molecules as previously described. Kits further contain buffers, reagents, and other solutions required to practice the methods as described herein.

The compositions, kits and methods of the present invention find utility in, but are not limited to, uses in research for identifying molecules that participate, for example, in a particular cellular function, signaling pathway, and the like. Drug discovery and drug interactions are also applications of the present invention, such that drugs can be identified to, for example, inhibit or upregulate cellular functions associated with cancers and other diseases and disorders. The compositions, methods and kits of the present invention also find utility in diagnostics, for example, in identifying molecules for use in disease diagnosis, in identifying molecules that are associated with disease states, or identifying molecules that are indicative of a subject at risk of developing a disease.

All publications and patents mentioned in the present application are herein incorporated by reference. Various modification and variation of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A method for detecting molecules complexed with, or in proximity to, a target biomolecule comprising:
a) providing a sample with a target biomolecule,
b) adding to said sample an activatable molecule for association with said biomolecule,
c) applying an activator to said sample so as to activate said activatable molecule to provide modifications to molecules within proximity to said target biomolecule, and
d) detecting said modifications to said molecules to identify molecules complexed with, or in proximity to, said target biomolecule.

2. The method of claim 1, wherein said sample is a cell lysate, cell extract, cell, tissue, environmental sample, bodily fluid, cerebrospinal fluid, urine, blood, plasma, serum, saliva, or bone marrow.

3. The method of claim 1, wherein said target biomolecule is nuclear or cytoplasmic.

4. The method of claim 3, wherein said nuclear or cytoplasmic target biomolecule is from a mammal, a virus, or bacteria.

5. The method of claim 4, wherein said target biomolecule is a protein, a nucleic acid, a signal transduction component, a receptor, a transcription factor, a histone, an enzyme, a kinase, a phosphatase, a galactosidase, a nuclease, a protease, a polymerase, a transferase, a transcriptase, a ligase, a reporter enzyme, a protamine, a phosphoprotein, a mucoprotein, a chromoprotein, a lipoprotein, a nucleoprotein, a glycoprotein, a T-cell receptor, a proteoglycan, a cancer antigen, a tissue specific antigen, hormones, or a nutritional marker.

6. The method of claim 4, wherein said target biomolecule is DNA, cDNA, telomeric DNA, RNA, mRNA, hnRNA, miRNA, siRNA, dsRNA, or an oligonucleotide.

7. The method of claim 1, wherein said activatable molecule is a photosensitizer.

8. The method of claim 1, wherein said activatable molecule is further conjugated to a binding moiety wherein said binding moiety is in association with said target biomolecule.

9. The method of claim 8, wherein said binding moiety is an antibody, a receptor, a ligand, or an aptamer.

10. The method of claim 1, wherein said activator is energy, light, or a chemical.

11. The method of claim 1, wherein said modifications are creation of carbonyl groups, sulfur oxidation, tyrosine crosslinks, chlorination, nitrosation, hydroxylation, tryptophanyl modifications, hydroxyl derivatives of aliphatic amino acids, protein deamination, amino acid interconversions, amino acid oxidation adducts, glycoxidation adducts, cross-linking, aggregation, or peptide bond cleavage.

12. The method of claim 1, wherein molecules within proximity to said target biomolecule are within at least 25 angstroms, at least 50 angstroms, at least 75 angstroms, at least 100 angstroms, at least 150 angstroms, at least 200 angstroms of said target biomolecule.

13. The method of claim 1, wherein said detecting said modifications to said molecules complexed with, or in proximity to, said target biomolecule comprises chemical detection.

14. The method of claim 13, wherein said chemical detection comprises the derivitization of said modification with dinitrophenylhydrazine.

15. The method of claim 14, further comprises capturing the dinitrophenylhydrazine derivatized modified molecules with an antibody to dinitrophenylhydrazine.

16. The method of claim 15, wherein said captured molecules are detected by an immunological assay.

17. The method of claim 16, wherein said immunological assay is from a group consisting of enzyme linked immunosorbent assay, immunohistochemistry, immunocytochemistry and immunoblotting.

18. The method of claim 13, wherein said chemical detection comprises the derivitization of said modification with a biotinylating compound.

19. The method of claim 18, further comprising the capturing of the biotinylated derivatized modified molecules with streptavidin.

20. The method of claim 19, wherein said captured molecules are detected by colorimetry, fluorometry, or radiometry.

21. The method of claim 1, wherein said identifying comprises analysis by mass spectroscopy, nuclear magnetic resonance imaging, or sequencing.

22. The method of claim 21, wherein said mass spectroscopy is matrix-assisted laser desorption ionization time-of-flight mass spectrometry or liquid chromatography tandem mass spectrometry.

23. The method of claim 1, wherein said detecting said modifications further comprises reduction of said modifications by a reducing agent.

24. The method of claim 23, wherein said reducing agent is dithiothreitol or mercaptoethanol.

25. The method of claim 23, wherein the reduced modifications are detected by chemical detection.

26. The method of claim 23, wherein said chemical detection comprises the biotinylation of said reduced modifications with a biotinylating compound.

27. The method of claim 26, further comprising the capturing of the biotinylated modified biomolecules by streptavidin.

28. The method of claim 27, wherein said captured molecules are detected by colorimetry, fluorimetry, or radiometry.

29. A kit comprising:
a) an activatable molecule,
b) a compound reactive with carbonyl or sulfhydryl reactive groups, and
c) a compound capable of capturing the reactive compound.

30. The kit of claim 29, wherein said kit further comprises a system for performing an enzyme linked immunosorbent assay.
